(19) United States
(12) Patent Application Publication
(10) Pub. No.: US 2025/0339087 A1
(43) Pub. Date: Nov. 6, 2025

(54) SYSTEMS AND METHODS TO QUANTIFY BALANCE ABILITY

(71) Applicant: Massachusetts Institute of Technology, Cambridge, MA (US)

(72) Inventors: Kaymie Shiozawa, Cambridge, MA (US); Neville Hogan, Sudbury, MA (US); Max Burns, Logan, OH (US)

(73) Assignee: Massachusetts Institute of Technology, Cambridge, MA (US)

(21) Appl. No.: 19/198,562

(22) Filed: May 5, 2025

Related U.S. Application Data
(60) Provisional application No. 63/642,951, filed on May 6, 2024.
(60) Provisional application No. 63/692,390, filed on Sep. 9, 2024.

(51) Int. Cl. A61B 5/00

(52) U.S. Cl. CPC A61B 5/4023

(57) ABSTRACT

Described herein is the design and implementation of an instrumented user device to produce quantitative metrics of patient fall risk. In some embodiments, a regression algorithm is used to estimate postural sway velocity, which is an effective predictor of fall risk. The instrumented user device enables continuous patient monitoring outside of the clinic and could provide a long-term, quantitative measure of fall risk.

### CROSS-REFERENCE TO RELATED APPLICATIONS

This application claims the benefit under 35 U.S.C. § 119 of U.S. Provisional Patent Application No. 63/642,951 filed on May 6, 2024, and also of U.S. Provisional Patent Application No. 63/692,390 filed on Sep. 9, 2024, both of which are hereby incorporated by reference in their entireties.

### STATEMENT REGARDING FEDERALLY SPONSORED RESEARCH

N/A

### BACKGROUND

In the US, those above 65 are expected to outnumber those under 18 by 2034, posing a unique set of public health challenges. In light of this emerging societal concern, the Center for Disease Control and Prevention has been raising awareness for “Healthy Aging” and encouraging the “development and maintenance of optimal physical, mental, and social well-being and function in older adults.” Among the most common impairments that prevent healthy aging in older adults are balance disorders that severely affect personal mobility.

### SUMMARY

Devices that assess human static balance have significant practical importance. These instruments may serve as a method to quantitatively diagnose individual patients and develop customized treatment plans, which may eventually lead to the development of customized assistive and/or rehabilitative technologies.

One form of quantitative assessment of human balance has been to apply external perturbations to the subject and measure their motion and/or force trajectories using high-precision instrumentation or motion-capture systems. The perturbations are usually applied via a custom-manufactured balance platform, such as NEUROCOM'S BALANCEMASTER or BERTEC'S COMPUTERIZED DYNAMIC POSTUROGRAPHY (CDP/IVR), that has been installed in a clinic or laboratory. However, perturbation-based analysis has several limitations. First, humans are highly adaptive. Their behavior in a laboratory setting, where they are subjected to artificial perturbations, may not be an accurate representation of the natural stance they exhibit on a day-to-day basis. Second, perturbations may be uncomfortable and even dangerous for balance-impaired populations. Furthermore, such experiments often involve big and expensive equipment that would be difficult to make widely available beyond specialized laboratories or clinics.

On the other hand, some clinical assessments not only test for perturbed balance performance but also evaluate quiet stance. Examples of such tests are the Berg Balance Test or the MiniBEST Test. However, these tests include subjective measures, such as “noticeable instability,” and coarse categories of performance, such as “normal,” “moderate,” and “severe” ability to complete certain tasks. These measures are difficult to interpret, as there may be a large variation of balance abilities subsumed within each category. Furthermore, since these clinical analyses are administered by clinicians, a physical therapist or a licensed person would need to be present to conduct them.

Finally, recent work has found postural sway analysis to yield effective predictions of fall risk and thus balance ability. Postural sway analysis is traditionally performed with a force plate, which measures the forces exerted by the patient's foot in a variety of conditions. The point of application of those forces is known as the center of pressure. In particular, the velocity of the center of pressure under different conditions has been found to correlate well with fall risk and age-related balance decline. However, due to the costly nature of the force plates that enable postural sway analyses, these tests can likely only be conducted in a clinic or laboratory setting. Even if more cost-effective alternatives were available, the form factor of the force plate would only allow for measurements to be taken intermittently. In other words, state of the art balance assessment tools do not allow for “on the go” measurements. There is evidently a need for quantitative, continuous methods of balance ability measurement.

This disclosure provides a continuous, portable alternative to the conventional systems to quantify human quiet balance ability. This system includes sensors that measure axial force, grip pressure, and angular and translational acceleration and velocity. The sensors can be mounted on a walking cane or other mobility aid, on another type of user device, or directly on the user. Such sensor data can be collected and processed to quantify static balance ability using methods disclosed herein. The sensors' outputs can be processed to estimate the subject's sway velocity, a strong predictor of fall risk, using a custom linear regression algorithm. In some embodiments, for each user, the mobility aid or other user device can be at first calibrated by subjecting the user to varying levels of balance challenge and measuring the “true” sway velocity using a force plate. Then, the user device can begin to estimate sway velocity without the need of a force plate. The methodology and device are validated against eight healthy, young participants who are tasked to balance in four balance conditions, and results show that sway velocity can be estimated with an average Variance Accounted For of 0.73. Since sway velocity is a more effective indicator of patient fall risk than traditional clinical balance assessments, disclosed devices, systems, and techniques provide an effective tool for continuously assessing patient fall risk.

This disclosure provides support for the use of an instrumented user device to produce quantitative measures of patient body sway and estimates of the Romberg balance quotient. A Romberg quotient is computed by dividing a measure of balance when a subject's eyes are closed, by the same measure when eyes are open. The ratio can be used to identify abnormal increases in instability when vision is removed, which can indicate poor proprioception. Romberg quotients are generally greater than one, with exact values varying based on the balance measure used. Prior work has shown that sway velocity or sway path length Romberg quotients effectively classify populations with various balance-affecting conditions. A sway path Romberg quotient over 2.0 may indicate a proprioceptive deficit; meanwhile, for adults without balance issues, the quotient is expected to be 1.2±0.3. Postural sway velocity and Romberg quotients can be combined for fall risk prediction.

Described herein is a design and validation of an instrumented user device with a regression algorithm used to estimate postural sway velocity in static balance. It is demonstrated herein that motion and force sensors mounted on a user device (or directly on a user) can be sufficient for accurately estimating the user's sway velocity. It is further demonstrated that Romberg quotients that strongly correlate with subjects' true sway velocity Romberg quotients can be computed using an instrumented user device. To demonstrate these points, a number of healthy young subjects and subjects over 65 (e.g., eight subjects in each group) can be evaluated under multiple balance conditions (e.g., four balance conditions) while using the instrumented user device. The data gathered from these experiments can be processed and used to train and test a regression algorithm to estimate sway velocity. It is shown that this method can produce effective estimates of postural sway velocity, with an average R² value of 0.73 for the younger subjects, and 0.47 for the older subjects. Results also suggested that hand motion is crucial to making effective sway velocity estimates. Romberg quotients computed from features related to hand motion are also found to correlate strongly with sway velocity Romberg quotients for each older subject. It is shown herein that the disclosed structures and techniques provide an instrumented mobility aid capable of estimating postural sway velocity and Romberg quotients.

In contrast to existing methods, disclosed systems and techniques do not require specialized personnel and complicated protocols. Rather, disclosed embodiments can provide for a device that resembles a typical walking cane or other mobility aid that a user can carry around with them in many settings including rough terrain, stairs, and slopes. Since the instrumented user device can look and feel similar to a regular cane that many balance-challenged adults use today, these users may simply switch out their normal cane with a disclosed instrumented user device to begin continuously monitoring their balance ability anywhere they go. Moreover, since the device can measure postural sway during static balance, external perturbation is not required for balance assessment.

Users may benefit from the ability to quantitatively assess the progression of their own balance health. This feature may be important for elders aging in their own homes, especially older women who are at high risk of fall-induced bone fracture. The instrumented user device can interface with Apple Health (for iOS users) or Google Fit (for Android users) to combine the quantified stability measure with the activity measurements tracked on a phone or a smart watch and display it in a user-friendly manner.

Clinicians may benefit from the quantitative measures provided by the system because it may serve as a new standard of diagnosis and help them to determine a customized treatment plan. The continuous nature of the measurements will enable health practitioners to understand patients' behaviors and challenges beyond the clinic. Finally, engineers who develop assistive devices (e.g., exoskeletal robots) may also use this system to quantify the change in balance performance with and without an assistive device. The data obtained can further be used to improve those devices.

While certain embodiments of the present disclosure may be described in the context of an instrumented mobility aid and more particularly an instrumented cane, the general concepts and techniques sought to be protected herein can be employed in various other systems and devices to quantity balance ability. For example, techniques described herein for estimating values such as sway velocity using portable motion can generally be used by any user device to which sensors (e.g., motion sensors and force sensors) are mounted, or even in configurations where the sensors are mounted on the user.

According to one aspect of the present disclosure, a system comprises: a user device having one or more sensors configured to generate data indicative of at least: orientation of the user device, movement of the user device, and forces exerted on the user device; and a processor configured to receive the data generated by the sensors and to quantify balance ability of a user by applying the received data as input to a regression model.

In some embodiments, the one or more sensors include at least one inertial measurement unit (IMU) configured to generate the data at least in part, the generated data indicative of at least: linear acceleration of the user device, angular velocity of the user device, and orientation of the user device. In some embodiments, the one or more sensors include one or more force-sensitive resistors (FSRs) integrated into a handle of the user device. In some embodiments, the one or more sensors include a load cell incorporated into a base of the user device to measure force applied along a shaft of the user device. In some embodiments, the regression model is a linear regression model.

In some embodiments, the processor is configured to quantify the balance ability of the user using one or more features generated from the received data, wherein the input to the regression model includes the one or more features. In some embodiments, the processor is configured to generate each of the one or more features by generating a raw data vector and applying a method to the raw data vector, wherein the raw data vector is one of: X Acceleration, aₓ, Y Acceleration, a_{y}, Z Acceleration, a_{z}, X Angular Velocity, ωₓ, Y Angular Velocity, ω_{y}, Z Angular Velocity, ω_{z}, Axial Force, F, Tilt Angle, θₜᵢₗₜ, Acceleration Magnitude, a_{mag}, X-Y Acceleration, a_{xy}, Tilt Angular Velocity, ωₜᵢₗₜ, or Angular Velocity Magnitude, ω_{mag}. The method can be one of: Mean, Median, Minimum, Maximum, Range, Interquartile Range, Skewness, Kurtosis, Standard Deviation, Mean Absolute Deviation, or Energy. In some embodiments, the one or more features include all combinations of the enumerated raw data vectors and methods. In some embodiments, the one or more features includes at least one of: ωₜᵢₗₜ Mean; ωₜᵢₗₜ Median; ω_{mag} Median; ω_{y} Mean Absolute Deviation; ω_{y} Interquartile Range; ω_{y} Mean Interquartile Range; Sway Velocity Mean; aₓ Mean; aₓ Median; and θₜᵢₗₜ Mean. In some embodiments, the processor is configured to select the one or more features by identifying and selecting sets of features that correlated closely with sway velocity and/or a balance ability measure, while penalizing features that correlated closely with one another.

According to another aspect of the present disclosure, a method comprises: receiving data generated by one or more sensors of a user device, the data indicating orientation of, movement of, and forces exerted on the user device; generating one or more features from the received data; and quantifying balance ability of a user of the user device using the one or more features.

In some embodiments, quantifying the balance ability of the user includes: providing the one or more features as input to a regression model; and quantifying the balance ability of a user based at least in part on output of the regression model. In some embodiments, the method can further comprises selecting the one or more features by identifying sets of features that correlated closely with sway velocity and/or a balance ability measure, while penalizing features that correlated closely with one another. In various other embodiments, the method can include features such as those described above with embodiments of the system.

It should be appreciated that individual elements of different embodiments described herein may be combined to form other embodiments not specifically set forth above. Various elements, which are described in the context of a single embodiment, may also be provided separately or in any suitable sub-combination. It should also be appreciated that other embodiments not specifically described herein are also within the scope of the following claims.

### BRIEF DESCRIPTION OF THE DRAWINGS

The manner of making and using the disclosed subject matter may be appreciated by reference to the detailed description in connection with the drawings, in which like reference numerals identify like elements.

FIG. 1 shows an example of an instrumented user device that can be used to quantify balance ability, according to some embodiments of the present disclosure.

FIG. 1A shows an example of a load cell mechanism that can be provided within an instrumented user device, according to some embodiments.

FIG. 2 illustrates measurements that can be collected by sensors of an instrumented user device, according to some embodiments.

FIGS. 3 and 4 illustrate an experimental study and protocol used to validate disclosed techniques and systems for quantifying balance ability using an instrumented user device.

FIG. 5 is a flow diagram illustrating a process for computing and evaluating the sway velocity regression model that can be used to quantify balance ability using an instrumented user device, according to some embodiments.

FIGS. 6A and 6B are representative plots of the estimation of sway velocity magnitude for two subject cohorts in an experiment of an instrumented user device as disclosed herein.

FIG. 7 illustrates results of conducting analysis of variances (ANOVA) for two subject cohorts in an experiment of an instrumented user device as disclosed herein.

FIG. 8 is a plot showing a comparison between Romberg quotients computed from mean sway velocity and mean ωₜᵢₗₜ for older subjects in a shoulder-width stance, in an experiment of an instrumented user device as disclosed herein.

FIG. 9 is a block diagram of a processing device on which methods and processes disclosed herein can be implemented, according to some embodiments of the disclosure.

The drawings are not necessarily to scale, or inclusive of all elements of a system, emphasis instead generally being placed upon illustrating the concepts, techniques, and systems sought to be protected herein.

### DETAILED DESCRIPTION

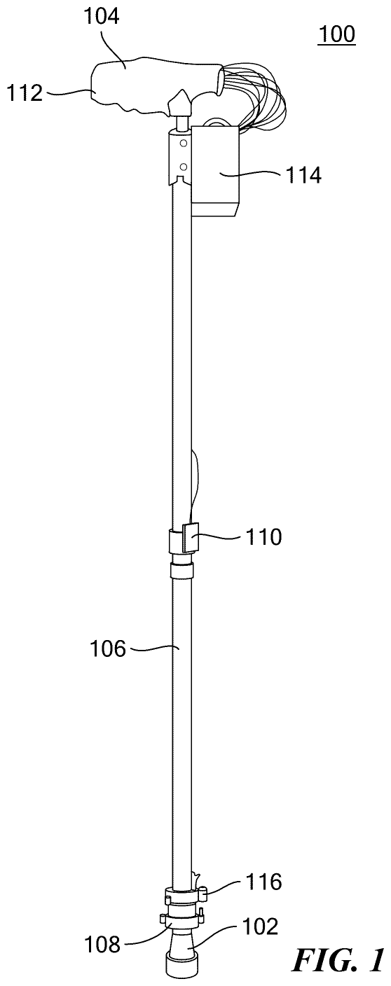

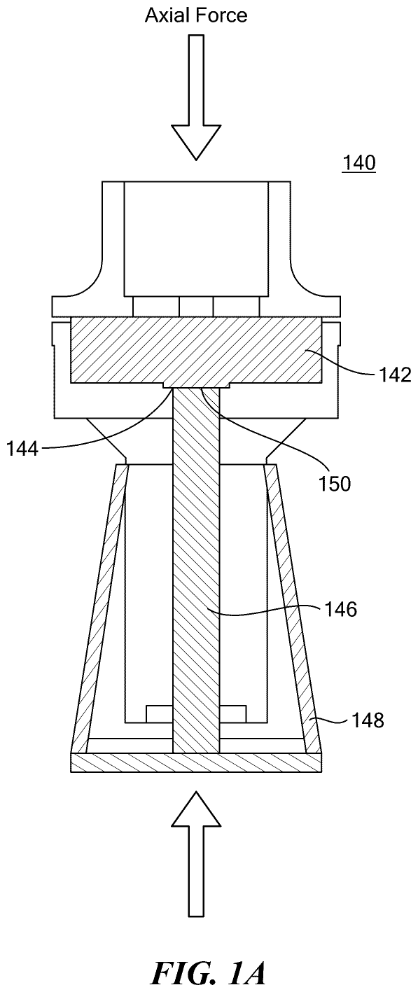

FIG. 1 shows an example of an instrumented user device 100 (e.g., a cane) that can be used to quantify balance ability, according to some embodiments of the present disclosure.

Illustrative user device 100 includes a base 102 and a handle 104 attached to opposite ends of a shaft 106. A load cell mechanism 108 (or “load cell mechanism”) can be incorporated into a base 102 of device 100 to measure axial force, meaning force applied along the shaft 106 of the user device. In some examples, load cell mechanism 108 can be installed into a ferrule of device 100. An inertial measurement unit (IMU) 110 can be attached to the shaft 106, or otherwise provided on the user device 100, to measure angular and translational motion of the user device 100 (e.g., linear acceleration, angular velocity, and orientation of the device). In some examples, IMU 110 can be positioned between base 102 and handle 104 along the length of the shaft. One or more force-sensitive resistors (FSRs) 112 can be integrated into the handle 104 to measure grip pressure. The sensors 108, 110, 112 shown and described are merely illustrative, and other types or combinations of sensors may be used. For example, in some embodiments the FSRs 112 may be omitted. In some examples, user device 100 can be a commercially available walking cane or other mobility aid onto which sensors 108, 110, 112 are mounted or otherwise incorporated. In some examples, the sensors 108, 110, 112 can be mounted directly to a user.

A microprocessor 114 can be provided on the user device 100 (e.g., attached to shaft 106) with input channels for receiving sensor data generated by the various sensors 108, 110, 112. In some examples, one or more of the sensors 108, 110, 112 can be connected to microprocessor 114 via wires that run along the length of the shaft 106. In some examples, one or more of the sensors 108, 110, 112 can be wirelessly connected to microprocessor 114. In some examples, microprocessor 114 may be configured to process sensor data (i.e., perform “onboard” or “local” processing). In some examples, microprocessor 114 may be configured to transmit sensor data to an external computing device for processing, using a wired (e.g., a USB Serial connection) or wireless data link. In some examples, user device 100 can include a storage means (e.g., flash memory) to which microprocessor 114 can store sensor data collected from sensors 108, 110, 112. The stored sensor data can then be downloaded/transmitted for “offline” processing. In some examples, microprocessor 114 can be an ARDUINO LEONARDO microprocessor. In some examples, microprocessor 114 can collect sensor data with a sample rate of 50 Hz, a baseline rate for human activity recognition.

In some examples, user device 100 can include a wireless transmitter configured to wireless transmit stored/collected sensor data to a remote processing system, e.g., a cloud computing system or other remote system hosting one or more applications that utilize processing techniques disclosed herein to quantity balance ability. The transmitter may be integrated into microprocessor 114, for example. One or more batteries (not shown) may be provided on the user device 100 and connected to power the microprocessor 114 and/or one or more of the sensors 108, 110, 112 for a period of hours, days, weeks, months, etc. Thus, user device 100 can be configured as a self-contained device capable of collecting, storing, and/or transmitting sensor data on a periodic or continuous basis (e.g., at a desired sampling frequency of 50 Hz, for example).

For correct cane use, it is suggested that only 15% of the user's weight should be supported by the cane. In some examples, load cell mechanism 108 can have a range selected to encompass approximately 30% of a standard 75 kg subject's body weight. Too small a range may lead to breakage of the load cell for large exerted force, and too large a range may sacrifice precision. In some examples, load cell mechanism 108 may be selected to have a range of 0-250 N. In some examples, a 250 lbf load cell force sensor manufactured by TE CONNECTIVITY MEASUREMENT SPECIALTIES (Model FC2311-0000-0250-L), with ±0.1 N resolution, ±0.1 N precision, and ±1% accuracy, may be used for load cell mechanism 108. In some examples, an amplifier 116 may be provided to amplify the signal generated by load cell mechanism 108 (i.e., load cell mechanism 108 may be indirectly connected to microprocessor 114 via amplifier 116). In some examples, amplifier 116 may be provided as a SPARKFUN QWIIC SCALE amplifier (Model NAU7802).

To measure grip pressure without disrupting the original shape of the device handle 104, relatively small FSRs 112 can be installed at strategic locations of the handle 104. In some examples, FSRs 112 can include a series of 0.5″ FSR 402 (Model SEN-09375), with 0.1 N to 10 N range, continuous resolution, and ±6%.

In some examples, to capture the device's motion, IMU 110 can be provided as a 9-axis IMU and, more particularly, as a SPARKFUN VR IMU BREAKOUT (Model SEN-14686), with 8 g accelerometer range, 0.3 m/s² accelerometer accuracy, 2000 ⁰/s gyroscope range, and 3.1 ⁰/s gyroscope accuracy.

In some examples, microprocessor 114 can be configured to execute software that records sensor data. In some examples, microprocessor 114 can obtain readings from IMU 110 as quaternions, 3-axis gyroscope measurements, and 3-axis acceleration measurements. In some examples, microprocessor 114 can convert an analog signal from load cell mechanism 108 to force (N) measurements. An initial reading may be made with the user device held vertically and lifted off the ground, and then subtracted from subsequent readings to “zero” the force; however, this process may be omitted for convenience. In some examples, microprocessor 114 can receive analog signals from FSRs 112 and record the analog values thereof.

An instrumented user device for quantifying balance ability, according to the present disclosure, can include any of the features and concepts described in K. S. Shiozawa, “Towards the Development of an Adaptive Rehabilitative Device,” M. S thesis, Massachusetts Institute of Technology, 2021, which is hereby incorporated by reference in its entirety.

FIG. 1A shows an example of a load cell mechanism 140 that can be provided within an instrumented user device, such as user device 100 of FIG. 1. The illustrative load cell mechanism 140 includes a load cell 142, a dowel pin 146, a ferrule 148, and a contact 150. Dowel pin 146 can be arranged to transfer axial force applied to the device at the base of the ferrule 148 to a localized area 144 (or “hub”) on the load cell. In some examples, dowel pin 146 can be provided as a steel pin (e.g., a 2-inch steel pin). Dowel pin 146 can be encased in a mechanism that allows for the pin to press upwards into the load cell 142 as ferrule 148 compresses.

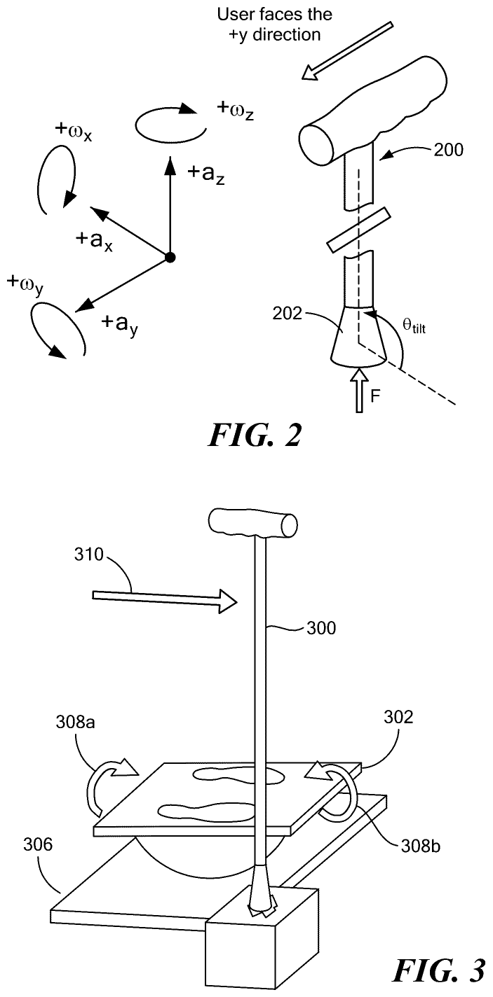

FIG. 2 shows an overview of sensors that can be mounted on a user device and used to quantify balance ability. Linear acceleration (aₓ, a_{y}, a_{z}), angular velocity (ωₓ, ω_{y}, ω_{z}), and orientation (θₜᵢₗₜ) of the handle can be collected from a 9-axis IMU provided on an instrumented user device 200. Axial force (F) can be collected from a load cell 202 inside of the device's base (or “tip”). The raw data vectors gathered from the user device and their respective directions are indicated in FIG. 2. In some examples, multiple data vectors can be computed based on the data acquired using the sensors, e.g., using software written in Python or another programming language. For example, the five data vectors outlined in Table I can be computed. The data vectors in Table I are merely illustrative and are not intended to be limiting. Additional and/or alternative data vectors may be computed using collected sensor data and used to quality balance ability, in keeping with the general concepts and techniques described herein. Table II, discussed below, shows other examples of data vectors that may be computed.

TABLE I
DATA VECTORS COMPUTED WITH COLLECTED DATA

| Computed Data Vector | Formula |
|---|---|
| Acceleration Magnitude, a_{mag} | ${a}_{mag} = \sqrt{{a}_{x}^{2} + {a}_{y}^{2} + {a}_{z}^{2}}$ |
| X-Y Acceleration, a_{xy} | ${a}_{xy} = \sqrt{{a}_{x}^{2} + {a}_{y}^{2}}$ |
| Angular Velocity Magnitude, ω_{mag} | ${ω}_{mag} = \sqrt{{ω}_{x}^{2} + {ω}_{y}^{2} + {ω}_{z}^{2}}$ |
| Tilt Angular Velocity, ωₜᵢₗₜ | ${ω}_{tilt} = \sqrt{{ω}_{x}^{2} + {ω}_{y}^{2}}$ |
| Tilt Angle, θₜᵢₗₜ | Quaternion Conversion |

The computed data vectors and the raw data vectors can be used for quantitative measurement of a user's static stability (e.g., balance ability) such as by using techniques described below.

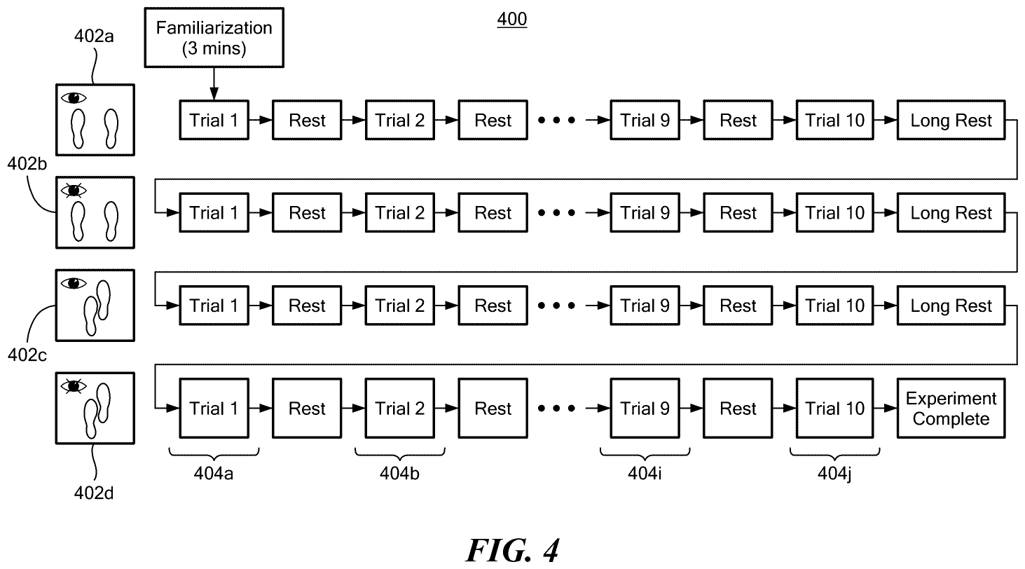

Turning to FIG. 3, to validate that an instrumented user device (e.g., user device 100 of FIG. 1) can be used to accurately estimate postural sway velocity, young healthy subjects can participate in a balance experiment with an instrumented user device 300. In practice, however, patients may participate in a training session with the instrumented user device, and a custom model can be computed for that individual. As more data is collected through the use of the instrumented user device, the model can be continuously updated as well.

In one experimental study, a first cohort comprising a number of young unimpaired human subjects (e.g., eight subjects) can be recruited, between the ages of 19 and 49 (e.g., average age of 25.4±9.8 years). All participating subjects may be inexperienced with using a cane or other user device and not report any neurological or muscular issues which affect their balance. Subjects may vary in height (1.65±0.09 m) and weight (65.3±13.2 kg) and exercise for an average of 7.1±4.5 hours per week, for example.

In the experiment, subjects in the first cohort (“younger cohort”) can stand on top of an unstable balance board 302, which is placed on top of a force plate 304 (e.g., a Kistler force plate). The subjects may face in a direction 310, as shown. The balance board may be free to rotate within the subjects' sagittal plane, allowing them to rock forward and back, as shown by arrows 308a, 308b indicating direction of free rotation.

A second cohort (“older cohort”) comprising a number of older subjects (e.g., eight individuals over the age of 65) can also be recruited to participate in a similar balance experiment to validate the instrumented device's ability to quantify balance measures of the target population. Only community-ambulating individuals, who are able to walk a certain distance (e.g., ten meters) without assistance and stand in place for a number of minutes (e.g., five minutes), may be considered eligible for this study. Their average age may be 80.7±9.6 years. Subjects of different balance abilities may be selected. For example, six subjects may not be affected by any self-reported balance conditions. One may have suffered some form of stroke in the past five years, and one may have previously been diagnosed with viral encephalitis. Subjects can vary in height (1.61±0.12 m) and weight (67.3±7.6 kg) and exercise for an average of 6.3±2.5 hours per week, for example. In contrast to the younger cohort, subjects in the older cohort may stand directly on the force plate. That is, unstable balance board 302 of FIG. 3 may be omitted for older subjects.

Center of pressure (CoP) data can be collected from the force plate 306 and used to calculate a user's sway velocity, which corresponds to the average speed of the subject's CoP. Sway velocity can be calculated by computing the absolute path length of the center of pressure and dividing by the time elapsed. Sway velocity calculated using a force plate or similar device is referred to herein as the “true” sway velocity, in contrast to “estimated” sway velocity which is calculated using only device-mounted sensors (e.g., some combination of sensors 108, 110, 112 of FIG. 1). In some embodiments, true sway velocity can be used to train a predictive model of estimated sway velocity, as discussed further below. In other words, force plate 306 may be used during a training phase. Once the training phase is complete, the trained model can be used to estimate sway velocity based only on data collected from the device-mounted sensors, thereby resulting in a system that allows for accurately estimating sway velocity of a user using only the user device 300 (and without the need for force plate 306).

During the experiment 400 depicted in FIG. 4, subjects can be asked to assume two stances, the first with their feet shoulder-width apart, and the second in the half-tandem orientation, where feet are close together and slightly offset in the sagittal plane. Subjects complete both stances with eyes open and then closed, yielding four conditions 402a-d. Other conditions may be used. In general, the number of types of conditions can be selected to create a high variance in stability.

In some cases, a different number of trials for each condition can be performed by each subject, based on subject ability and fatigue. This approach may be used, in particular, for the older cohort. Table II shows an example of the number of trials and conditions that may be used for eight (8) different subjects in the older cohort, and also indicates the numbers that may be used for all subjects in the younger cohort.

TABLE II
NUMBER OF COMPLETED TRIALS
BY SUBJECT, BY CONDITION

| | Shoulder-Width | | Half-Tandem | | |
|---|---|---|---|---|---|
| Subject | Eyes | Eyes | Eyes | Eyes | Total Data |
| Number | Open | Closed | Open | Closed | Windows |
| 1 | 6 | 6 | 6 | 5 | 138 |
| 2 | 6 | 6 | 6 | 6 | 144 |
| 3 | 10 | 10 | 10 | 3 | 198 |
| 4 | 6 | 6 | 6 | 6 | 144 |
| 5 | 10 | 10 | 10 | 0 | 180 |
| 6 | 6 | 6 | 6 | 6 | 144 |
| 7 | 6 | 6 | 6 | 6 | 144 |
| 8 | 6 | 6 | 6 | 6 | 144 |
| Younger | 10 | 10 | 10 | 10 | 240 |
| Cohort | | | | | |

A number of trials 404a, 404b, etc. (404 generally) can be conducted for each condition 402a-d, during which younger subjects can be instructed to focus on keeping the balance board as stable and level as possible while holding the instrumented user device, and older subjects can be instructed to remain as stable as possible. At the end of each condition 402a-d, subjects can be asked to rest for a period (e.g., two minutes or longer) to minimize the effect of fatigue. In some examples, ten trials 404a-j can be used to maximize the amount of data obtained, without reaching a point where subjects lost focus on the task or became fatigued. In some examples, each trial 404 can last approximately thirty-six (36) seconds. It is appreciated herein that transitioning between conditions too frequently may decrease subject consistency. Thus, four blocks of ten trials can be used, with a relatively long rest between each block. A trial length of about thirty-six (36) seconds can be selected to maximize the number of data windows which could be obtained from each trial, without exhausting subjects and compromising consistency. To prevent subjects from lifting the user device in the air, subjects can be instructed that their balance will be evaluated by the force plate on which the balance board is placed (e.g., force plate 306 of FIG. 3).

For each trial, center of pressure data can be collected from the force plate at a sampling frequency of 50 Hz, for example. Then, the average speed of the subject's center of pressure, sway velocity, can be calculated over a window of time (e.g., a ten second window of time). This computation can be performed based upon only the center of pressure of the subject's feet, without including force on the user device, as the force on the user device (an average body weight borne of 3.08±2.07% for the younger cohort, 2.26±1.18% for the older) may be sufficiently low. Sway velocity can be calculated by computing the absolute path length of the center of pressure and dividing by the time elapsed. Sway velocity is an effective predictor of fall risk and suitable to serve as the “true” stability metric that can be estimated using an instrumented user device. The true sway velocity calculated using a force plate can be used to train a predictive model of sway velocity based on sensor data, as discussed next. The trained model can subsequently be used to quantify balance ability of an end user using only the instrumented user device (i.e., without requiring an end user to stand on a force plate).

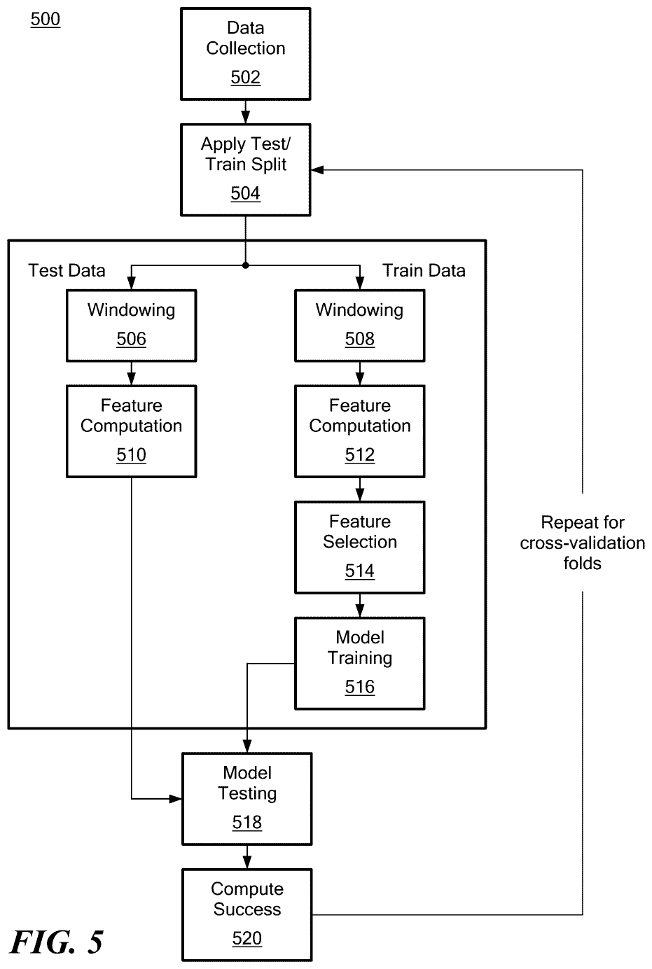

Turning to FIG. 5, a process 500 illustrates steps for computing and evaluating a sway velocity linear regression model, including preparation of data for use in the model. Process 500 can be used to train a model for estimating body sway velocity using motion and force data from one or more sensors mounted on a user device or directly on the user.

At step 502, raw data can be collected/recorded by an instrumented user device over multiple trials (e.g., forty trials as shown in FIG. 4) and multiple test subjects (e.g., eight test subjects). The raw data can include, for example, linear acceleration (aₓ, a_{y}, a_{z}), angular velocity (ωₓ, ω_{y}, ω_{z}), orientation (θₜᵢₗₜ), and axial force (F). In some examples, the raw data may be collected into one or more data vectors, referred to herein as “raw data vectors.”

At step 504, the data can be split into a training dataset and a testing dataset. For example, 65% of the data can be randomly assigned as the training set, while the other 35% can be designated as the testing set. A test-train split can be established to evaluate the predictive ability of the regression model. Stratified three-fold cross validation can be used to divide each subject's trials into three equal bins. For example, a given subject may perform six trials under each of the four balance challenges, so the stratification process can ensure that two trials from each condition are selected for each of the three folds. The stratification process can be used to avoid class imbalance between cross validation folds, which would interfere with model training due to the dissimilar test and train datasets.

Raw force plate data (e.g., CoP data) or true sway velocity data calculated therefrom can be used during the model training phase to compute the weights of the model that result in a good estimate of “true” sway velocity based on collected sensor data (motion and force data collected from sensors mounted on a user device). For example, raw force plate data and/or true sway velocity data can be included within the training dataset of process 500.

At steps 506 and 508, for each trial, a sliding window can be applied with overlap between windows. For example, a ten second sliding window with overlap (e.g., 48% or 50% overlap) may be used. The overlap and window length can be selected to maximize the number of data points produced per trial without causing overfitting. In one validation experiment, a sliding window can produce six windows for each 36-second trial, resulting in a maximum of 156 training windows and 84 testing windows per subject.

Window length and number of features used can be selected through a parameter sweep using, for example, a cloud-based supercomputing cluster. In some examples, window lengths between two (2) and seventeen (17) seconds and total feature counts between three (3) and sixteen (16) features can be examined in a 2-dimensional parameter sweep. In one particular example, a window length of ten (10) seconds and a feature count of ten (10) may be used to produce acceptable and stable R² values across subjects.

At steps 510 and 512, features can be extracted from the raw data recorded by the instrumented user device. Each feature can be computed by applying a statistical method to one window of raw data. For instance, the “mean axial force” feature can be computed by applying the mean method to a window of axial force data. In some examples, each method can be applied to each raw data vector such that 132 features, or 11 methods for each of the 12 raw data vectors, are produced per window. Feature computation can be performed for each window of time series data. Table III shows examples of functions that can be used for feature production, e.g., by applying the listed methods to the listed data vectors in various permutations. This process can be repeated multiple times (e.g., 288 times) to yield the results presented in this work.

In other embodiments, raw time-series data can be used directly for training instead of or in addition to computing statistics (e.g., mean, median, minimum, etc.) of each data vector.

TABLE III
DATA VECTORS AND APPLIED METHODS
USED TO PRODUCE FEATURES

| Data Vectors | Methods |
|---|---|
| X Acceleration, aₓ | Mean |
| Y Acceleration, a_{y} | Median |
| Z Acceleration, a_{z} | Minimum |
| X Angular Velocity, ωₓ | Maximum |
| Y Angular Velocity, ω_{y} | Range |
| Z Angular Velocity, ω_{z} | Interquartile Range |
| Axial Force, F | Skewness |
| Tilt Angle, θₜᵢₗₜ | Kurtosis |
| Acceleration Magnitude, a_{mag} | Standard Deviation |
| X-Y Acceleration, a_{xy} | Mean Absolute Deviation |
| Tilt Angular Velocity, ωₜᵢₗₜ | Energy |
| Angular Velocity Magnitude, ω_{mag} | |

Of note, the following are data vectors that can be obtained directly from the IMU and axial force sensor, referred to herein as “raw data vectors”:
X acceleration
Y acceleration
Z acceleration
X angular velocity
Y angular velocity
Z angular velocity
Axial Force

The following are data vectors that can be computed from the above data vectors using the methods described in Table I, referred to herein as “computed data vectors”.
Tilt angle—using quaternion conversions
Acceleration magnitude— see formula in Table I
XY acceleration—see formula in Table I
Tilt angular velocity—see formula in Table I
Angular velocity magnitude—see formula in Table I

At step 514, feature selection can be performed on the training data. For each model, a feature selection process can be used to reduce the risk of overfitting and remove ineffective features. Feature selection can be performed with an optimizer configured to identify sets of features that correlated closely with sway velocity, while penalizing features that correlated closely with one another. This process can reduce redundancy between the selected features, while also ensuring that the features selected are strong predictors of sway velocity. Feature selection can be performed based upon only training data to prevent contamination of test data. In some examples, twelve features can be selected for use in linear regression. The feature selection process can be conducted using the training data from all subjects. Due to the random nature of the test-train split, slightly different feature sets may be selected during the repeated production of models.

At step 516, the training data for each subject can be used to train one or more linear regression models. The training of each subject's model can be conducted using just the individual subject's training dataset. In testing, no consistent trends are observed in the residuals, indicating that linear regression is suitable for this purpose.

At steps 518 and 520, each model can be applied to the corresponding subject's test dataset, and the estimated sway velocity can be compared to the true sway velocity. An R² value can be computed for the test data of each subject. R² values closer to the maximum of one (1) indicate accurate estimation of sway velocity. In one example, due to the random nature of the test-train split, 288 linear regression models can be prepared for each subject, using 3-fold cross validation, to determine an average RMSE and R² value for each individual. Cross validation can be used to reduce the variability of the model output and ensure consistent model evaluation.

The features selected can vary between models, due to the variability of the test-train split. Additionally, the feature selection process can discourage redundant features, and so two features which are closely related to one another (and for the purposes of the model are effectively interchangeable) may be underrepresented in any count of feature occurrence. This variability can make compilation of common features for the purpose of reporting results difficult, so a different approach may be undertaken. For example, the Pearson correlation coefficient between sway velocity magnitude and each feature can be computed. A feature with an absolute correlation of greater than 0.8 for the younger cohort, or 0.5 for the older cohort, can be considered a “strong” feature for that subject. This process can be repeated for each subject, and the strong features can be recorded.

To determine an effective selection of window length and number of features, a parameter sweep can be performed using a computing system (e.g., a cloud computing system). In some examples, window lengths between 2 and 17 seconds, and total feature counts between 3 and 16 features can be examined in a 2-dimensional parameter sweep. In testing, a window length of 10 seconds and a feature count of 12 are found to produce acceptable and stable values across subjects. This parameter sweep can be conducted to find the most suitable window length and number of features for the purposes of testing, but it would not be used with a patient as the window length and number of features will be kept constant.

To examine the ability of the instrumented user device to distinguish between balance conditions, three-way analyses of variances (ANOVAs) can be conducted for each feature, separately for the younger and older cohorts. Stance (shoulder-width or half-tandem), eye status (open or closed), and subject number can be used as the independent variables, and feature values can be used as the dependent variables. The f-statistics and p-values can be recorded for stance and eye status, and then sorted to find the features which provide the greatest distinction between conditions. Of note, while ANOVAs are used for the purposes of the validation study, they are not intended to be used in a device.

A Romberg quotient is the ratio of any measure of balance with eyes closed to eyes opened. The quotients can be obtained for the older subject cohort from the instrumented user device. For this analysis, no test-train split may be applied, and data windows can be assigned a chronological index on a per-subject basis. Romberg quotients can be computed for all features, by performing element-wise division of the eyes closed feature vector by the eyes open feature vector of the same index. The correlation of these feature Romberg quotients to the true Romberg quotient (computed via the sway velocity measured by the force plate) can be evaluated for each subject. This analysis can be confined to the shoulder-width stance condition. The quotient with the highest correlation can be reported.

TABLE IV
R2 AND RMSE RESULTS: 250 ITERATIONS OF
CROSS-VALIDATION

| | | Sway Velocity | ML Sway | AP Sway |
|---|---|---|---|---|
| | | Magnitude | Velocity | Velocity |
| Younger | R2 | 0.73 ± 0.04 | 0.73 ± 0.04 | 0.57 ± 0.05 |
| Cohort | $RMSE ( \frac{mm}{s} )$ | 8.29 ± 0.51 | 6.43 ± 0.40 | 5.60 ± 0.36 |
| Older | R2 | 0.47 ± 0.09 | 0.45 ± 0.09 | 0.36 ± 0.12 |
| Cohort | $RMSE ( \frac{mm}{s} )$ | 5.88 ± 0.37 | 4.71 ± 0.31 | 3.80 ± 0.24 |

Table IV summarizes the R² and RMSE values for each component of sway velocity, averaged across all subjects. For the younger cohort, an average R² value of 0.73±0.04 is found between the linear regression result and the mean magnitude of sway velocity. For the cohort of subjects over 65, an average R² value of 0.47±0.09 is observed for sway velocity magnitude. For the younger subjects, the highest average R² for sway velocity magnitude of an individual subject is 0.86±0.05, while the lowest is 0.60±0.12. For the older cohort, the highest average R² for a subject is 0.67±0.13, and the lowest 0.34±0.31.

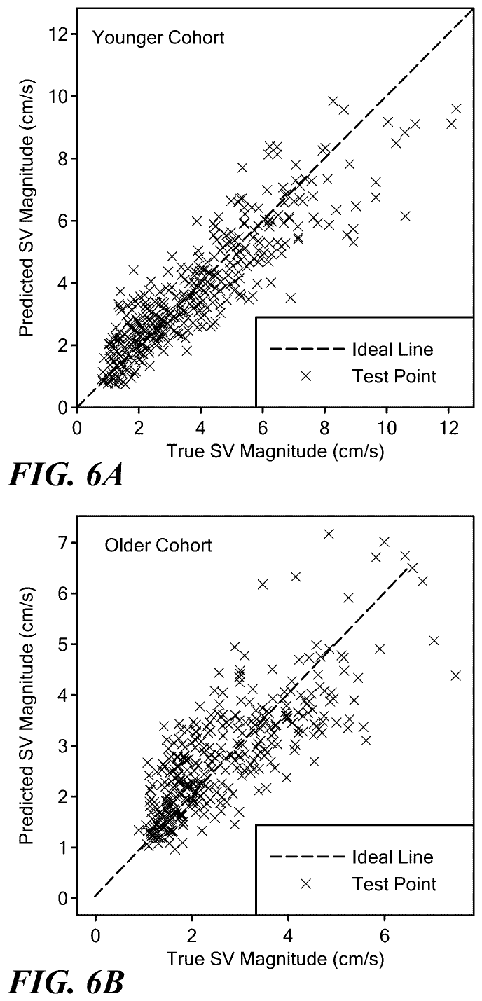

FIGS. 6A and 6B demonstrates example models for the older and younger cohorts, which compared the estimated sway velocity magnitude of each data window to the true sway velocity magnitude for the younger and older cohorts respectively. FIGS. 6A and 6B are representative plots of the estimation of sway velocity magnitude for the two subject cohorts, with FIG. 6A showing data from the younger cohort and FIG. 6B showing data from the older cohort. Each point is from one 10 s window of test data from a subject. The diagonal line represents the ideal output, where the estimation or prediction error is zero.

TABLE V
“STRONG” FEATURES FOUND IN YOUNGER SUBJECTS

| | Subjects with | Avg. Correlation |
|---|---|---|
| Feature | Correlation >0.8 | Sway Vel. |
| ωₜᵢₗₜ Mean | 8 | 0.88 ± 0.05 |
| ω_{mag} Median | 8 | 0.87 ± 0.05 |
| ωₜᵢₗₜ Median | 7 | 0.87 ± 0.05 |
| ω_{y} Mean Absolute Deviation | 7 | 0.85 ± 0.05 |
| ω_{y} Interquartile Range | 7 | 0.85 ± 0.05 |

TABLE VI
“STRONG” FEATURES FOUND IN OLDER SUBJECTS

| | Subjects with | Avg. Correlation |
|---|---|---|
| Feature | Correlation >0.8 | Sway Vel. |
| ωₜᵢₗₜ Mean | 8 | 0.88 ± 0.05 |
| ω_{mag} Median | 8 | 0.87 ± 0.05 |
| ωₜᵢₗₜ Median | 7 | 0.87 ± 0.05 |
| ω_{y} Mean Absolute Deviation | 7 | 0.85 ± 0.05 |
| ω_{y} Interquartile Range | 7 | 0.85 ± 0.05 |

Tables V and VI summarize the features that displayed high Pearson's correlation with sway velocity magnitude for the two cohorts. Notably, features which described the magnitude and variability of ωₜᵢₗₜ (the angular velocity about the instrumented device's base) are particularly well correlated with sway velocity magnitude for both subject cohorts.

TABLE VII
RESULTS OF THREE-WAY ANOVA
FOR YOUNGER COHORT

| | |
|---|---|
| Feature | F-Statistic |

Eye Status

| | |
|---|---|
| Sway Velocity Mean | 3142.6 |
| ω_{y} Mean Absolute Deviation | 2539.9 |
| ωₜᵢₗₜ Median | 2520.5 |
| ω_{y} Interquartile Range | 2508.6 |

Stance

| | |
|---|---|
| aₓ Mean | 143.6 |
| θₜᵢₗₜ Minimum | 138.3 |
| aₓ Median | 130.3 |
| Sway Velocity Mean | 114.9 |

TABLE VIII
RESULTS OF THREE-WAY ANOVA FOR OLDER COHORT

| | |
|---|---|
| Feature | F-Statistic |

Eye Status

| | |
|---|---|
| Sway Velocity Mean | 260.9 |
| a_{mag} Median | 136.6 |
| ωₜᵢₗₜ Median | 130.6 |
| ω_{y} Interquartile Range | 129.0 |

Stance

| | |
|---|---|
| ω_{y} Mean Absolute Deviation | 215.6 |
| ωₜᵢₗₜ Median | 208.2 |
| Fₐₓ Standard Deviation | 195.3 |
| Sway Velocity Mean | 90.1 |

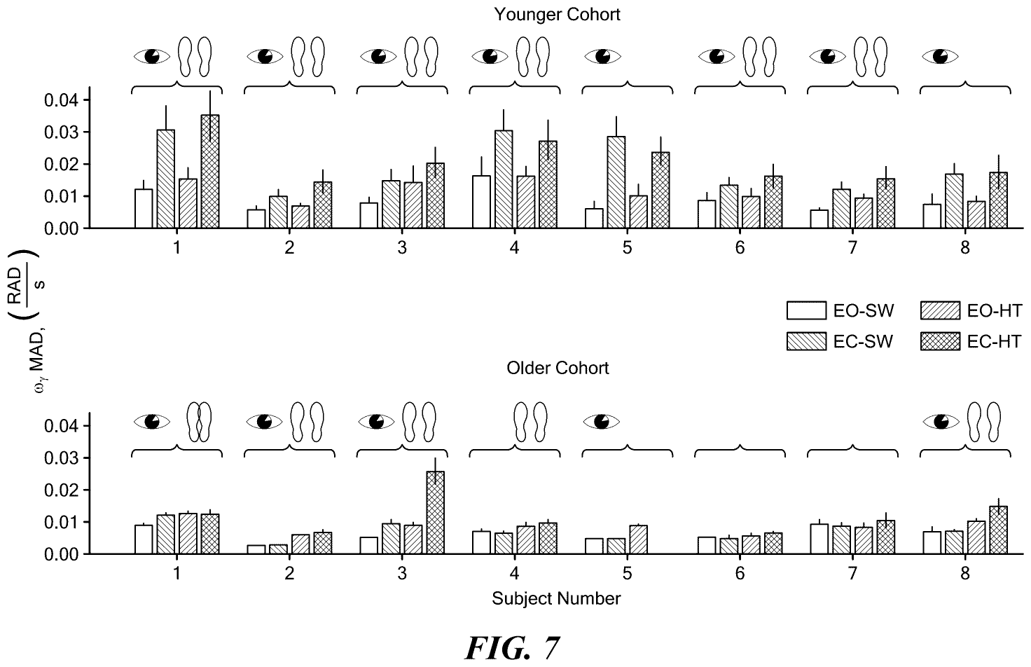

Among each cohort, the three features with the largest f-statistic for eye status and stance determined through three-way ANOVA are summarized in Tables VII and VIII. These features can be primarily computed from acceleration or angular velocity signals, with the exception of the standard deviation of axial force for the stance conditions of the older cohort. Because the mean absolute deviation of the Y angular velocity, ω_{y}, displayed the greatest degree of significance for the subjects' eye status, that feature is selected to be plotted in FIG. 7 to demonstrate an example of the two-way ANOVA.

FIG. 7 shows an example of the results of conducting ANOVA. The mean absolute deviation (MAD) of the Y angular velocity, ω_{y}, can be computed for each data window of each subject, and a two-way ANOVA can be conducted for each subject. The top row shows data from the younger subject cohort, while the bottom row shows data from the older cohort. EC and EO refer to “eyes open” and “eyes closed” respectively. SW and HT refer to “shoulder-width” and “half-tandem” respectively. The presence of eye and footprint icons above a subject's data indicate significance for eye status and stance respectively, found through two-way ANOVA.

The two-way ANOVA revealed that although each feature displayed significant differences for eye status and stance when all data are considered, for individual subjects, this may not always be true. For the younger subject cohort, all features shown in Table VII are found to be significant for eye status in the two-way ANOVAs.

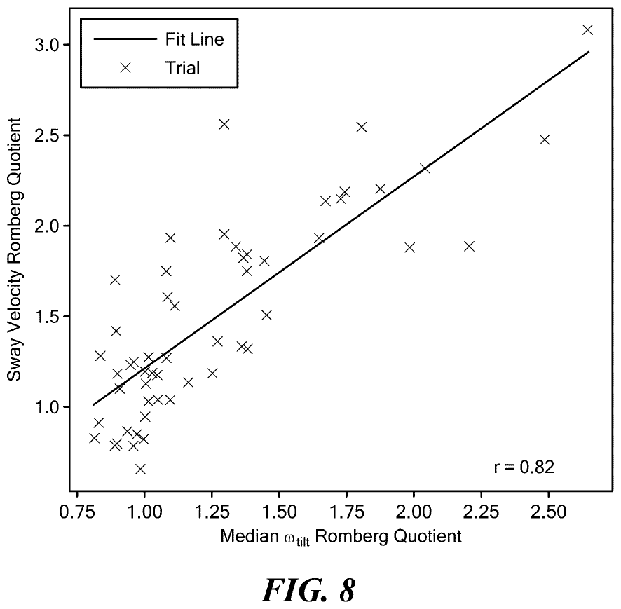

For the older cohort, ωₜᵢₗₜ correlated most strongly (r-value of 0.82) with the true Romberg quotient in the shoulder-width stance. This Romberg quotient correlation across all older subjects are shown in FIG. 8.

FIG. 8 shows a comparison between Romberg quotients computed from mean sway velocity and mean ωₜᵢₗₜ for older subjects in the shoulder-width stance. In the figure, each “x” icon represents the quotient computed from an eyes-open and eyes-closed trial for one subject. Data from all older subjects is displayed and used to compute the correlation. A Pearson correlation of r=0.82 is observed, with p=4e-14. A line of best fit, of which the slope is 1.06±0.10 and the intercept is 0.15±0.04, is shown.

Postural sway velocity is a measure that can be used to estimate fall risk. Described herein is the use of an instrumented user device to estimate a user's sway velocity and Romberg quotient during quiet stance. Results described herein demonstrate that a user device's instrumentation of motion and force sensors are sufficient to estimate sway velocity across all participants. Furthermore, aspects of the participants' hand motion are closely tied to body sway for all subjects. Romberg quotients computed from instrumented user device metrics are found to have strong correlation with true Romberg quotients computed from sway velocity.

This work demonstrated that the outputs of sensors onboard an instrumented user device can reliably estimate a user's postural sway velocity and Romberg quotient. The device's motion data can be used for accurately estimating these measures for both healthy young subjects and those over 65.

Although the nature of falls are complex, postural sway velocity and the Romberg quotient are both important markers of a patient's balance health and can be used in models to predict future fall risk. The Romberg quotient, in particular, can indicate poor proprioception and overreliance on visual feedback for balance.

Disclosed systems and techniques can be used to provide an instrumented user device intended to be an at-home monitoring device which assesses a patient's stability over the course of months or years, rather than the few instances during which they visit the clinic. To effectively estimate a patient's sway velocity and assess fall risk, a clinician would first collect a set of standard training data by asking the patient to balance in place on a force plate (which measures sway velocity) while using the instrumented user device. Based on these trials, a linear regression model is customized for this individual and used to estimate sway velocity when the patient stands still in their daily life after they leave the clinic. This methodology allows for at-home, continuous assessment of fall risk, which would be a powerful tool for clinicians to identify changes or long-term trends in their patients' health. At-home monitoring can provide advance notice regarding a patient's physical condition and could prevent falls before they occur. The model presented herein can inform a physician in the development of a treatment plan or whether intervention is necessary.

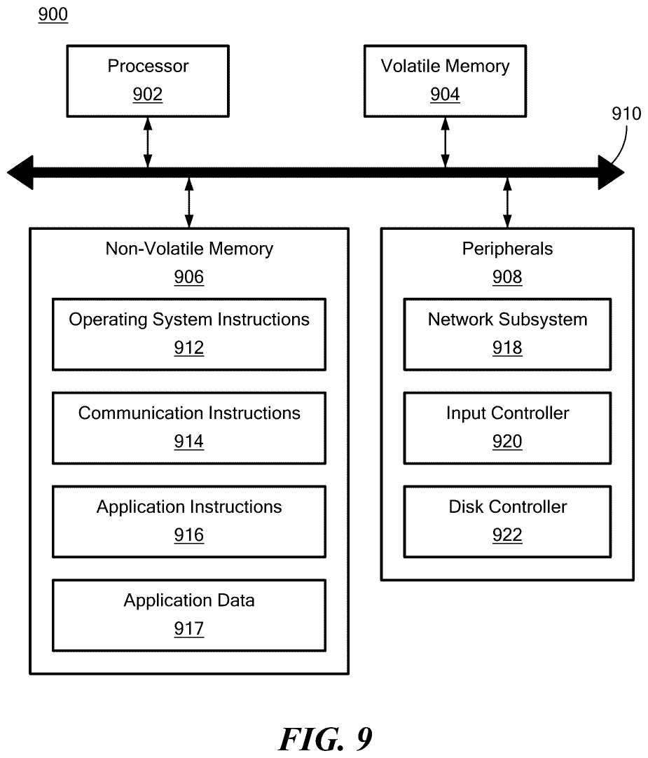

FIG. 9 shows an illustrative processing device 900 that may implement various features and processes as described. For example, processing device 900 may be used to collect sensor data from an instrumented user device, to compute data vectors from the collected data, and to train and apply a linear regression model for determining balance ability using the computed data vectors. Illustrative processing device 900 may be used for onboard and/or external processing such as discussed above in the context of FIG. 1.

Processing device 900 may include one or more processors 902, volatile memory 904, non-volatile memory 906, and one or more peripherals 908. These components may be interconnected by one or more computer buses 910.

Processor(s) 902 may use any known processor technology, including but not limited to graphics processors and multi-core processors. Suitable processors for the execution of a program of instructions may include, by way of example, both general and special purpose microprocessors, and the sole processor or one of multiple processors or cores, of any kind of computer. Bus 910 may be any known internal or external bus technology, including but not limited to ISA, EISA, PCI, PCI Express, NuBus, USB, Serial ATA or FireWire. Volatile memory 904 may include, for example, SDRAM. Processor 902 may receive instructions and data from a read-only memory or a random-access memory or both. The essential elements of a computer may include a processor for executing instructions and one or more memories for storing instructions and data.

Non-volatile memory 906 may include by way of example semiconductor memory devices, such as EPROM, EEPROM, and flash memory devices; magnetic disks such as internal hard disks and removable disks; magneto-optical disks; and CD-ROM and DVD-ROM disks. Non-volatile memory 906 may store various computer instructions including operating system instructions 912, communication instructions 914, application instructions 916, and application data 917. Operating system instructions 912 may include instructions for implementing an operating system (e.g., Mac OS®, Windows®, or Linux). The operating system may be multi-user, multiprocessing, multitasking, multithreading, real-time, and the like. Communication instructions 914 may include network communications instructions, for example, software for implementing communication protocols, such as TCP/IP, HTTP, Ethernet, telephony, etc.

Peripherals 908 may be included within the processing device 900 or operatively coupled to communicate with the processing device 900. Peripherals 908 may include, for example, network interfaces 918, input devices 920, and storage devices 922. Network interfaces may include, for example, an Ethernet or Wi-Fi adapter. Input devices 920 may be any known input device technology, including but not limited to a keyboard (including a virtual keyboard), mouse, trackball, and touch-sensitive pad or display. Storage devices 922 may include one or more mass storage devices for storing data files; such devices include magnetic disks, such as internal hard disks and removable disks; magneto-optical disks; and optical disks.

The system can perform processing, at least in part, via a computer program product, (e.g., in a machine-readable storage device), for execution by, or to control the operation of, data processing apparatus (e.g., a programmable processor, a computer, or multiple computers). Each such program may be implemented in a high-level procedural or object-oriented programming language to communicate with a computer system. However, the programs may be implemented in assembly or machine language. The language may be a compiled or an interpreted language and it may be deployed in any form, including as a stand-alone program or as a module, component, subroutine, or other unit suitable for use in a computing environment. A computer program may be deployed to be executed on one computer or on multiple computers at one site or distributed across multiple sites and interconnected by a communication network. A computer program may be stored on a storage medium or device (e.g., CD-ROM, hard disk, or magnetic diskette) that is readable by a general or special purpose programmable computer for configuring and operating the computer when the storage medium or device is read by the computer. Processing may also be implemented as a machine-readable storage medium, configured with a computer program, where upon execution, instructions in the computer program cause the computer to operate. The program logic may be run on a physical or virtual processor. The program logic may be run across one or more physical or virtual processors.

As used herein, the term “processor” is used to describe electronic circuitry that performs a function, an operation, or a sequence of operations. The function, operation, or sequence of operations can be hard coded into the electronic circuit or soft coded by way of instructions held in a memory device. The function, operation, or sequence of operations can be performed using digital values or using analog signals. In some embodiments, a processor can be embodied in an application specific integrated circuit (ASIC), which can be an analog ASIC or a digital ASIC, in a microprocessor with associated program memory, in a digital signal processor (DSP), and/or in a discrete electronic circuit, which can be analog or digital. A processor can include internal processors or modules that perform portions of the function, operation, or sequence of operations. Similarly, a module can include internal processors or internal modules that perform portions of the function, operation, or sequence of operations of the module. A single processor or other unit may fulfill the functions of several means recited in the claims.

As used in the claims or elsewhere herein, the term “comprising” does not exclude other elements or steps, and the indefinite article “a” or “an” does not exclude a plurality.

The subject matter described herein can be implemented in digital electronic circuitry, or in computer software, firmware, or hardware, including the structural means disclosed herein and structural equivalents thereof, or in combinations of them. The subject matter described herein can be implemented as one or more computer program products, such as one or more computer programs tangibly embodied in an information carrier (e.g., in a machine-readable storage device), or embodied in a propagated signal, for execution by, or to control the operation of, data processing apparatus (e.g., a programmable processor, a computer, or multiple computers). A computer program (also known as a program, software, software application, or code) can be written in any form of programming language, including compiled or interpreted languages, and it can be deployed in any form, including as a stand-alone program or as a module, component, subroutine, or another unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub programs, or portions of code). A computer program can be deployed to be executed on one computer or on multiple computers at one site or distributed across multiple sites and interconnected by a communication network.

The processes and logic flows described in this disclosure, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read-only memory or a random-access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of nonvolatile memory, including by ways of example semiconductor memory devices, such as EPROM, EEPROM, flash memory device, or magnetic disks. The processor and the memory can be supplemented by, or incorporated in, special purpose logic circuitry.

A computer program may be stored/distributed on a suitable medium, such as an optical storage medium or a solid-state medium supplied together with or as part of other hardware, but may also be distributed in other forms, such as via the Internet or other wired or wireless telecommunication systems.

Various embodiments of the concepts systems and techniques are described herein with reference to the related drawings. Alternative embodiments can be devised without departing from the scope of the described concepts. It is noted that various connections and positional relationships (e.g., over, below, adjacent, etc.) are set forth between elements in the claims, detailed description, and drawings. These connections and/or positional relationships, unless specified otherwise, can be direct or indirect, and the claimed inventions are not intended to be limiting in this respect. Accordingly, a coupling/connection of entities can refer to either a direct or an indirect coupling/connection, and a positional relationship between entities can be a direct or indirect positional relationship. As an example of an indirect positional relationship, references in the present description to element or structure A coupled/connected to element or structure B include situations in which one or more intermediate elements or structures (e.g., element C) is provided between elements A and B regardless of whether the characteristics and functionalities of elements A and/or B are substantially changed by the intermediate element(s).

Furthermore, it should be appreciated that relative, directional or reference terms (e.g. such as “above,” “below,” “left,” “right,” “top,” “bottom,” “vertical,” “horizontal,” “front,” “back,” “rearward,” “forward,” etc.) and derivatives thereof are used only to promote clarity in the description of the figures. Such terms are not intended as, and should not be construed as, limiting. Such terms may simply be used to facilitate discussion of the drawings and may be used, where applicable, to promote clarity of description when dealing with relative relationships, particularly with respect to the illustrated embodiments. Such terms are not, however, intended to imply absolute relationships, positions, and/or orientations. For example, with respect to an object or structure, an “upper” or “top” surface can become a “lower” or “bottom” surface simply by turning the object over. Nevertheless, it is still the same surface and the object remains the same.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value. The term “substantially equal” may be used to refer to values that are within ±20% of one another in some embodiments, within ±10% of one another in some embodiments, within ±5% of one another in some embodiments, and yet within ±2% of one another in some embodiments.

In the foregoing detailed description, various features are grouped together in one or more individual embodiments for the purpose of streamlining the disclosure. This method of disclosure is not to be interpreted as reflecting an intention that each claim requires more features than are expressly recited therein. Rather, inventive aspects may lie in less than all features of each disclosed embodiment.

References in the disclosure to “one embodiment,” “an embodiment,” “some embodiments,” or variants of such phrases indicate that the embodiment(s) described can include a particular feature, structure, or characteristic, but every embodiment can include the particular feature, structure, or characteristic. Moreover, such phrases are not necessarily referring to the same embodiment(s). Further, when a particular feature, structure, or characteristic is described in connection knowledge of one skilled in the art to affect such feature, structure, or characteristic in connection with other embodiments whether or not explicitly described.

The disclosed subject matter is not limited in its application to the details of construction and to the arrangements of the components set forth in the detailed description or illustrated in the drawings. The disclosed subject matter is capable of other embodiments and of being practiced and carried out in various ways. As such, those skilled in the art will appreciate that the conception, upon which this disclosure is based, may readily be utilized as a basis for the designing of other structures, methods, and systems for carrying out the several purposes of the disclosed subject matter. Therefore, the claims should be regarded as including such equivalent constructions insofar as they do not depart from the spirit and scope of the disclosed subject matter.

Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter.

Other variations to the disclosed embodiments can be understood and effected by those skilled in the art in practicing the claimed invention, from a study of the drawings, the disclosure, and the appended claims.

The mere fact that certain measures are recited in mutually different dependent claims does not indicate that a combination of these measures cannot be used to obtain an advantage.

Any reference signs in the claims should not be construed as limiting the scope.

All publications and references cited herein are expressly incorporated herein by reference in their entirety.

1. A system comprising:
a user device having one or more sensors configured to generate data indicative of at least:
orientation of the user device,
movement of the user device, and
forces exerted on the user device; and
a processor configured to receive the data generated by the sensors and to quantify balance ability of a user by applying the received data as input to a regression model.

2. The system of claim 1, wherein the one or more sensors include at least one inertial measurement unit (IMU) configured to generate the data at least in part, the generated data indicative of at least:
linear acceleration of the user device,
angular velocity of the user device, and
orientation of the user device.

3. The system of claim 1, wherein the one or more sensors include one or more force-sensitive resistors (FSRs) integrated into a handle of the user device.

4. The system of claim 1, wherein the one or more sensors include a load cell incorporated into a base of the user device to measure force applied along a shaft of the user device.

5. The system of claim 1, wherein the processor is configured to quantify the balance ability of the user using one or more features generated from the received data, wherein the input to the regression model includes the one or more features.

6. The system of claim 5, wherein the processor is configured to generate each of the one or more features by generating a raw data vector and applying a method to the raw data vector, wherein the raw data vector is one of:
X Acceleration, aₓ,
Y Acceleration, a_{y},
Z Acceleration, a_{z},
X Angular Velocity, ωₓ,
Y Angular Velocity, ω_{y},
Z Angular Velocity, ω_{z},
Axial Force, F,
Tilt Angle, θₜᵢₗₜ,
Acceleration Magnitude, a_{mag},
X-Y Acceleration, a_{xy},
Tilt Angular Velocity, ωₜᵢₗₜ, or
Angular Velocity Magnitude, ω_{mag},
and the method is one of:
Mean,
Median,
Minimum,
Maximum,
Range,
Interquartile Range,
Skewness,
Kurtosis,
Standard Deviation,
Mean Absolute Deviation, or
Energy.

7. The system of claim 6, wherein the one or more features include all combinations of the enumerated raw data vectors and methods.

8. The system of claim 6, wherein the one or more features includes at least one of:
ωₜᵢₗₜ Mean;
ωₜᵢₗₜ Median;
ω_{mag} Median;
ω_{y} Mean Absolute Deviation;
ω_{y} Interquartile Range;
ω_{y} Mean Interquartile Range;
Sway Velocity Mean;
aₓ Mean;
aₓ Median; and
θₜᵢₗₜ Mean.

9. The system of claim 5, wherein the processor is configured to select the one or more features by identifying and selecting sets of features that correlated closely with sway velocity and/or a balance ability measure, while penalizing features that correlated closely with one another.

10. The system of claim 1 wherein regression model is a linear regression model.

11. A method comprising:
receiving data generated by one or more sensors of a user device, the data indicating orientation of, movement of, and forces exerted on the user device;
generating one or more features from the received data; and
quantifying balance ability of a user of the user device using the one or more features.

12. The method of claim 11 wherein quantifying the balance ability of the user includes:
providing the one or more features as input to a regression model; and
quantifying the balance ability of a user based at least in part on output of the regression model.

13. The method of claim 12 wherein the regression model is a linear regression model.

14. The method of claim 11, wherein the one or more sensors include an inertial measurement unit (IMU) configured to generate data indicating linear acceleration, angular velocity, and orientation of the user device.

15. The method of claim 11, wherein the one or more sensors include one or more force-sensitive resistors (FSRs) integrated into a handle of the user device.

16. The method of claim 11, wherein the one or more sensors include a load cell incorporated into a base of the user device to measure force applied along a shaft of the user device.

17. The method of claim 11, generating the one or more features from the received data includes, for each feature, generating a raw data vector and applying a method to the raw data vector, wherein the raw data vector is one of:
X Acceleration, aₓ,
Y Acceleration, a_{y},
Z Acceleration, a_{z},
X Angular Velocity, ωₓ,
Y Angular Velocity, ω_{y},
Z Angular Velocity, ω_{z},
Axial Force, F,
Tilt Angle, θₜᵢₗₜ,
Acceleration Magnitude, a_{mag},
X-Y Acceleration, a_{xy},
Tilt Angular Velocity, ωₜᵢₗₜ, or
Angular Velocity Magnitude, ω_{mag},
and the method is one of:
Mean,
Median,
Minimum,
Maximum,
Range,
Interquartile Range,
Skewness,
Kurtosis,
Standard Deviation,
Mean Absolute Deviation, or
Energy.

18. The method of claim 17, wherein the one or more features include all combinations of the enumerated raw data vectors and methods.

19. The method of claim 17, wherein the one or more features includes at least one of:
ωₜᵢₗₜ Mean;
ωₜᵢₗₜ Median;
ω_{mag} Median;
ω_{y} Mean Absolute Deviation;
ω_{y} Interquartile Range;
ω_{y} Mean Interquartile Range;
Sway Velocity Mean;
aₓ Mean;
aₓ Median; and
θₜᵢₗₜ Mean.

20. The method of claim 11 further comprising selecting the one or more features by identifying sets of features that correlated closely with sway velocity and/or a balance ability measure, while penalizing features that correlated closely with one another.
